(19) United States
(12) Patent Application Publication
(10) Pub. No.: US 2010/0022848 A1
(43) Pub. Date: Jan. 28, 2010

(54) PHYSIOLOGICAL STATUS MONITORING/POSITIONING SYSTEM

(75) Inventors: YANG-HAN LEE, Taipei (TW); WEI CHIEN, Taipei (TW); WEI-CHEN LEE, Taipei (TW)

Correspondence Address: WPAT, PC;INTELLECTUAL PROPERTY ATTORNEYS, 7225 BEVERLY ST., ANNANDALE, VA, 22003, US

(73) Assignee: IMRISS TECHNOLOGY CORPORATION, TAIPEI (TW)

(21) Appl. No.: 12/199,174

(22) Filed: Aug. 27, 2008

(30) Foreign Application Priority Data
Jul. 25, 2008 (TW) 097128456

(51) Int. Cl. A61B 5/00

(52) U.S. Cl. 600/300

(57) ABSTRACT

A physiological status monitoring/positioning system is disclosed, which comprises: a physiological status sensing device, being configured with at least a sensing unit and a signal transmitting unit to be used for detecting and transmitting a physiological attribute of a living entity; a positioning device, being configured with a second signal transceiving unit to be used for transmitting a second signal to a positioning system where the location of the living entity is identified; a calculation device, being configured with a first signal transceiving unit for receiving the physiological attribute while converting the same into a bio index to be transmitted; a signal transceiver, for transmitting/receiving the bio index; and a display device, for displaying the identified location of the living entity as well as the bio index. With the aforesaid system, the physiological status of the living entity as well as its location can be monitored at the same time so that as soon as an abnormal physiological status of the living entity is detected, an immediate action can be taken in response to the emergency.

### FIELD OF THE INVENTION

The present invention relates to a physiological status monitoring/positioning system, and more particularly, to system capable of monitoring the physiological status of a living entity as well as its location at the same time.

### BACKGROUND OF THE INVENTION

Outdoor activities, such as taking a walking tour or mountain climbing, are common recreations during weekend or holiday. Most of us are like to gang up with friends, relatives, or even fellow workers to go hiking in holidays as it is not just good for health, but also for improving relationship as well.

For preventing partners from getting lost or traveling in a wrong route during the hiking, it is common to communicate with each other of the same group by walkie-talkie or cellular phone, or even to use global position system (GPS) devices for guidance. However, Such communication enabled by the use of walkie-talkie or cellular phone as well as the ability to acknowledge each other the location of all the personnel in the same hiking group by the use of GPS devices could not alert the occurring of any abnormal physiological status if some/any hiker is not feeling well. Mountain accident usually happens to those who get lost in the hiking or injured so that it is important to have a way for informing his/her fellow hikers that he/she is lost or injured can be critical for life saving. Such getting lost condition or health emergency can also be applied to family pets like dogs, since the lose of one's loving pet can be a huge mental disaster for its owner.

Therefore, it is in need of a physiological status monitoring/positioning system capable of monitoring the physiological status of a living entity as well as its location at the same time, by which as soon as an abnormal physiological status of the living entity is detected, an immediate action can be taken in response to the emergency since the location of the living entity is known.

### SUMMARY OF THE INVENTION

The object of the present invention is to provide a physiological status monitoring/positioning system capable of monitoring the physiological status of a living entity as well as its location at the same time, by which the disadvantages of those conventional positioning system are overcame as they can only identify the location of the living entity but has not way of knowing the physical status of the same.

To achieve the above object, the present invention provides a physiological status monitoring/positioning system, comprising: a physiological status sensing device, being configured with at least a sensing unit and a signal transmitting unit to be used for detecting and transmitting a physiological attribute of a living entity; a positioning device, being configured with a second signal transceiving unit to be used for transmitting a second signal to a positioning system where the location of the living entity is identified; a calculation device, being configured with a first signal transceiving unit for receiving the physiological attribute while converting the same into a bio index to be transmitted; a signal transceiver, for transmitting/receiving the bio index; and a display device, for displaying the identified location of the living entity as well as the bio index.

With the aforesaid system, the physiological status of the living entity as well as its location can be monitored at the same time so that as soon as an abnormal physiological status of the living entity is detected, an immediate action can be taken in response to the emergency.

Further scope of applicability of the present application will become more apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given herein below and the accompanying drawings which are given by way of illustration only, and thus are not limitative of the present invention and wherein:

FIG. 1 shows a physiological status monitoring/positioning system according to an exemplary embodiment of the invention.

FIG. 2 shows a physiological status sensing device being embedded in a handlebar of a bicycle according to an exemplary embodiment of the invention.

FIG. 3 is a schematic diagram showing the configuration of a signal transceiver and a display unit of a physiological status monitoring/positioning system in a handheld electronic device according to an exemplary embodiment of the invention.

FIG. 4 is a schematic diagram showing the configuration of a signal transceiver and a display unit of a physiological status monitoring/positioning system in an electric calculation apparatus according to an exemplary embodiment of the invention.

FIG. 5 is a schematic diagram showing the configuration of a physiological status monitoring/positioning apparatus in an animal collar according to an exemplary embodiment of the invention.

FIG. 6 is a schematic diagram showing how the bio indexes are being displayed on the display device of the invention.

### DESCRIPTION OF THE EXEMPLARY EMBODIMENTS

For your esteemed members of reviewing committee to further understand and recognize the fulfilled functions and structural characteristics of the invention, several exemplary embodiments cooperating with detailed description are presented as the follows.

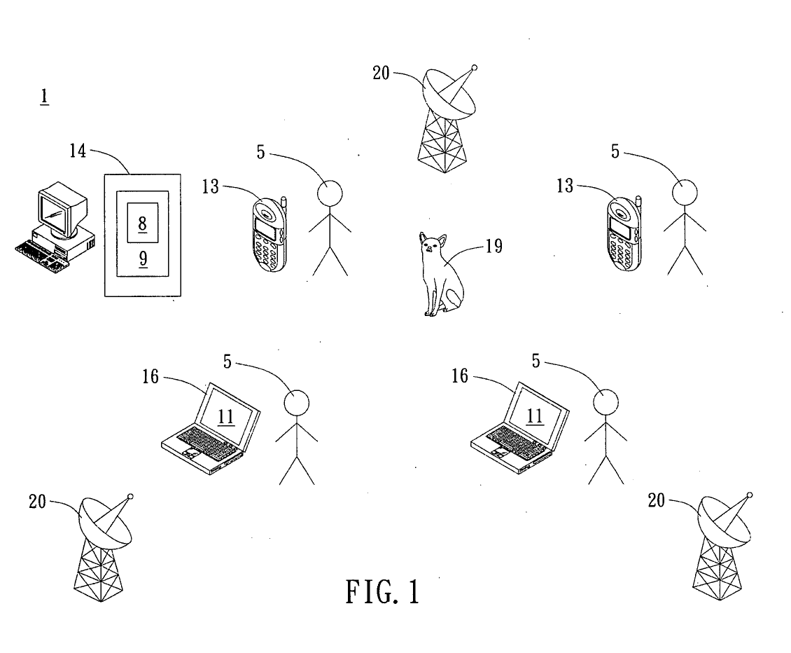

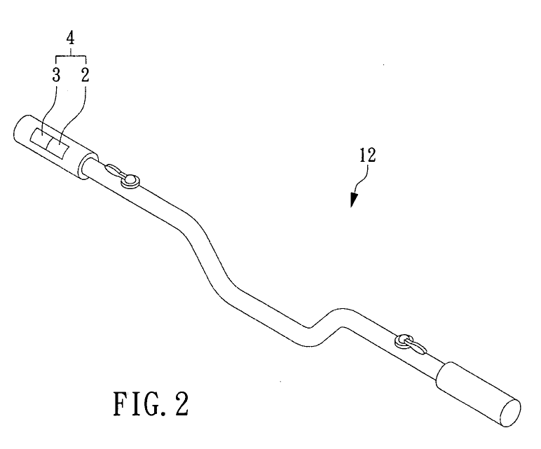

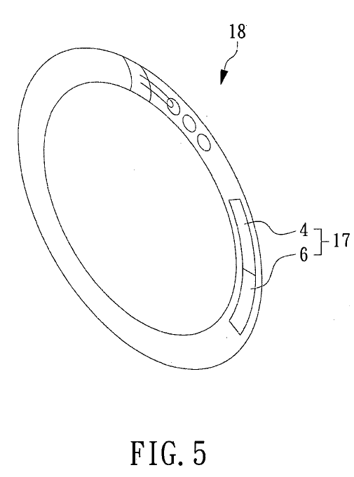

Please refer to FIG. 1 and FIG. 2, which respectively show a physiological status monitoring/positioning system according to an exemplary embodiment of the invention and a physiological status sensing device of the invention being embedded in a handlebar of a bicycle. In FIG. 1, the physiological status monitoring/positioning system 1 comprises: a physiological status sensing device 4, being configured with at least a sensing unit 2 and a signal transmitting unit 3 to be used for detecting and transmitting a physiological attribute of a living entity; a positioning device 6, being configured with a second signal transceiving unit 7 to be used for transmitting a second signal to a positioning system where the location of the living entity is identified; a calculation device 9, being configured with a first signal transceiving unit 8 for receiving the physiological attribute while converting the same into a bio index to be transmitted; a signal transceiver 10, for transmitting/receiving the bio index; and a display device 11, for displaying the identified location of the living entity as well as the bio index. In the embodiment shown in FIG. 2, the physiological status sensing device 4 is mounted on one end or both ends of a handlebar 12 of a bicycle, which can be powered by a power supply such as a battery mounted on the bicycle, or by power harnessed from the movement of the bicycle. Thus, when the bicycle is riding by a living entity like a user 5, he/she will hold on to the handlebar 12 by one hand or both hands by which the sensing unit 2 will be able to detect the physiological attribute of the living entity. It is noted that the physiological attribute can be a value of physiological voltage, but is not limited thereby. Such detected physiological attribute is then being transmitted by the signal transmitting unit 3, which can be an antenna, for enabling the same to be received by the first signal transceiving unit 8 (such as an antenna) of the calculation device 9 where it is being converted into the bio index which can be adapted to be displayed as a chart selected from the group consisting of: an electroencephalogram (EEG), an electrocardiogram (ECG), an electrogastrogram (EGG) and an electromyogram (EMG), but is not limited thereby. It is noted that the positioning device 6 is enabled to identify the location of the user 5 at the same time when the bio index is being generated. There can be two location identification methods, one of which is by the use of the second signal transceiving unit 7 to send a signal regarding to a coordinate to a position system like a GPS system at which the location of the living entity is determined. Another method is by the use of the second signal transceiving unit 7 to send a positioning signal, such as a caller ID signal of cellular phone, a BT signal or a ZigBee signal, to be received by more than one base stations of a position system by which the coordinate relating to the location of the living entity can be calculated and thus locate the living entity. For designing a compact system, the physiological sensing device 4 and the positioning device 6 can be integrated into a physiological status monitoring/positioning apparatus 17, as the one shown in FIG. 5. which is able to detect the physiological attribute of a living entity and the same time locate the position of the living entity. It is noted that the aforesaid physiological attribute, the coordinate and the bio index are transmitted through a communication network system, which can be a wireless communication system or a wireless network system, such as GPRS, 3G, 4G, WiFi, BT and ZigBee. However, it is possible to include a wired communication system in the communication network system. Moreover, although the aforesaid embodiment is exemplified by the use of a bicycle's handlebar, such physiological sensing device can be configured and integrated with any apparatuses accessible to the user 5, such as his/her backpack, driving wheel of his/her car, or even hats, which can be something that have no direct contact with the user since the physiological sensing device can be designed to detect the user's physiological attribute in a wireless sensing manner.

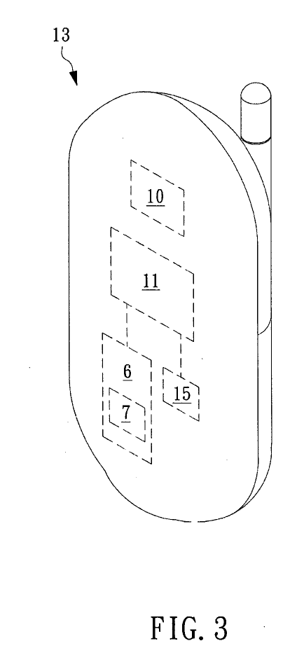

Please refer to FIG. 3, which is a schematic diagram showing the configuration of a signal transceiver and a display unit of a physiological status monitoring/positioning system in a handheld electronic device according to an exemplary embodiment of the invention. As shown in FIG. 3, the handheld electronic device 13 is substantially a cellular phone, but is not limited thereby. As the signal transceiver 10 and the display unit 11 is embedded in the handheld electronic device 13 while the calculation device 9 is configured in a center operation system 14, the physiological attribute detected by the sensing unit 2 is first being sent to the center operation system 14 through the communication network system where it is being converted into the bio index and then transmitted through the communication network system by the use of the first signal transceiving unit 8 so as to be received by the signal transceiver 10 of the handheld electronic device 13 for enabling the display device 11 to display the coordinate of the living entity as well as the bio index. It is noted that the signals relating to the coordinate and the bio index are transmitted in an alternative manner representing the alternative relationship between the coordinate and the bio index. In addition, the signals can be used in a physical analysis. In an exemplary embodiment, each and every sensing unit 2 used in the physiological status monitoring/positioning system of the invention can be identified individually and thus grouped by the center operation system 14 by the use of a specific identification method. As for the positioning signal, it is usually being transmitted by the second signal transmitting unit 7. However, it is common to integrate the signal transceiver 10 and the positioning device 6 into a chip for enabling the two to share an antenna for transmitting the positioning signal along with the signals of the bio index. In another preferred embodiment, the handheld electronic device 13 is designated as the positioning device 6, by which the signal emitted from the handheld electronic device 13 is treated as the positioning signal which can be received by a positioning system for location identification. Operationally, when a group of users 5 are riding bicycles together, it is common that they might not be able to see each other in the group as they are riding at different speeds. However, as each user 5 in the group had carried a handheld electronic device, the users 5 in the group are able to monitor the bio indexes and aware of the positions of other users by the communication of the communication network system. In addition, the handheld electronic device 13 is designed to form a closed circuit of Mesh or PtP with other handheld electronic devices 13 in the group in a manner that they are enabled to receive the coordinates and the bio indexes of other users 5 in the group directly from one another without having to communication through the communication network system and thus alleviate the load of the same. Nevertheless, since any user 5 in the group will not be able to keep an eye on the coordinates and the bio indexes of other users 5 in the group constantly as he/she is on to something, it is preferred to configured an alert unit 15 in the handheld electronic device 13 so as to be used for executing an alert operation as soon as the bio index exceeds a specific threshold value, and the alert operation can comprise the step of: enabling the cellular phone to alerting those other cellular phones in the communication network system in a manner such as calling out automatically to other cellular phones, or marking the cellular phone performing the alert operation as an eye-catching symbol on the display devices of those other cellular phones. It is noted that the alert unit may be designed to call out automatically to other cellular phones in the group automatically as soon as the bio index exceeds the specific threshold value, and the eye-catching symbol can be an outstanding colored mark or a flashing mark.

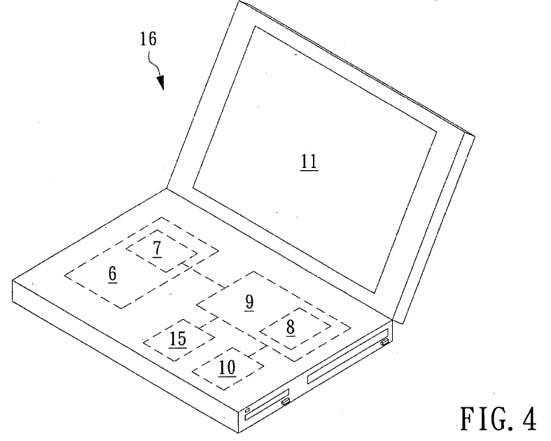

Please refer to FIG. 4, which is a schematic diagram showing the configuration of a signal transceiver and a display unit of a physiological status monitoring/positioning system in an electric calculation apparatus according to an exemplary embodiment of the invention. In FIG. 4, the electric calculation apparatus 16 is a notebook computer, however, it can be a desktop computer. The notebook computer is used as an illustration since it is becoming more and more commonplace, and is lighter, smaller and more powerful that it is possible to be integrated with cellular phone in the near future. Thus, in FIG. 4, the aforesaid calculation device 9 and the display device 11 is configured in the electric calculation apparatus 16 by that the physiological attribute and the coordinate are received by the first signal transceiving unit 8 through the communication network system and then the received physiological attribute is converted by the calculation device 9 into the bio index so as to be display on the display device 11. As for the positioning signal, it is usually being transmitted by the second signal transmitting unit 7. However, it is common to integrate the calculation device 9 and the positioning device 6 into a chip for enabling the two to share an antenna for transmitting the positioning signal along with the signals of the bio index. In another preferred embodiment, the electric calculation apparatus 16 is designated as the positioning device 6, by which the signal emitted from the electric calculation apparatus 16 is treated as the positioning signal which can be received by a positioning system for location identification. Operationally, the electric calculation apparatus 16 is acting as the monitor in the communication network system which is able to constantly monitor the bio indexes and coordinate of all the users 5 in the group as it is able to communicate with each and every handheld electronic devices 13 by the communication of the communication network system. Similarly, it is preferred to configured an alert unit 15 in the electric calculation apparatus 16 so as to be used for executing an alert operation as soon as the bio index exceeds a specific threshold value, and the alert operation can comprise the step of: enabling the electric calculation apparatus 16 to alerting those other electric calculation apparatuses 16 or even other handheld electronic devices 16 like cellular phones in the communication network system in a manner such as calling out automatically to other cellular phones, or marking the cellular phone performing the alert operation as an eye-catching symbol on the display devices of those other cellular phones. It is noted that the alert unit may be designed to call out automatically to other cellular phones in the group automatically as soon as the bio index exceeds the specific threshold value, and the eye-catching symbol can be an outstanding colored mark or a flashing mark. In addition, as the electric calculation apparatus 16 is designed with a capability for converting the physiological attribute into bio index, it can form a closed circuit with other electric calculation apparatuses 16 or handheld electronic devices 13 in the group in a manner that they are enabled to receive the coordinates and the bio indexes of other users 5 in the group directly from one another without having to communication through the communication network system and thus alleviate the load of the same.

Please refer to FIG. 5, which is a schematic diagram showing the configuration of a physiological status monitoring/positioning apparatus in an animal collar according to an exemplary embodiment of the invention. As the animal collar 18 embedded with the physiological status monitoring/positioning apparatus 17 can be worn by animals such as a pet cat or even a wild animal, the pet owner or an ecological observer is able to monitor the physical status as well as the location of the animal by the use of the aforesaid handheld electronic device or electric calculation apparatus through the communication of the communication network system. In addition, as the signals relating to the coordinate and the bio index are transmitted in an alternative manner representing the alternative relationship between the coordinate and the bio index, the signals can be very helpful especially for the ecological observer.

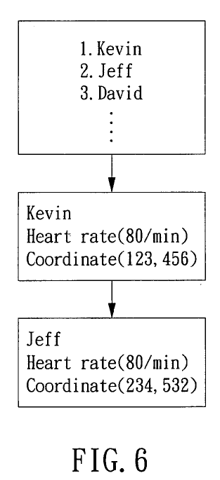

Please refer to FIG. 6, which is a schematic diagram showing how the bio indexes are being displayed on the display device of the invention. In FIG. 6, when a group of users are connected with each other by the use of the physiological status monitoring/positioning system of the invention, any user is able to obtain the information relating to who are currently in the group through the display of his/her display device. Moreover, by switching the display, the information relating to the coordinate and bio index of a specific user in the group can be displayed. It is noted that such display is not limited to be demonstrated in an literal manner, and thus can be demonstrated graphically by marking the locations of the users in the group in a map while enabling the symbols of the user, who are not feeling well, to flash or become red. Thereby, as soon as an abnormal physiological status of any user in the group is detected, an immediate action can be taken by other users in the group in response to the emergency.

From the above description, the present invention provides a physiological status monitoring/positioning system capable of monitoring the physiological status of a living entity as well as its location at the same time so that as soon as an abnormal physiological status of the living entity is detected, an immediate action can be taken in response to the emergency.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A physiological status monitoring/positioning system, comprising:
a physiological status sensing device, being configured with at least a sensing unit and a signal transmitting unit to be used for detecting and transmitting a physiological attribute of a living entity;
a positioning device, being configured with a second signal transceiving unit to be used for transmitting a second signal to a positioning system where the location of the living entity is identified;
a calculation device, being configured with a first signal transceiving unit for receiving the physiological attribute while converting the same into a bio index to be transmitted;
a signal transceiver, for transmitting/receiving the bio index; and
a display device, for displaying the identified location of the living entity as well as the bio index.

2. The system of claim 1, wherein the second signal specifies a coordinate in a coordinate system.

3. The system of claim 1, wherein the second signal is substantially a positioning signal.

4. The system of claim 2, wherein the physiological attribute, the coordinate and the bio index are transmitted through a communication network system.

5. The system of claim 3, wherein the physiological attribute, the coordinate and the bio index are transmitted through a communication network system.

6. The system of claim 4, wherein the signal transceiver and the display unit are configured in a handheld electronic device while enabling the signal transceiver to transmit/receive the coordinate and the bio index through the communication network system and thus to display the same on the display unit.

7. The system of claim 5, wherein the signal transceiver and the display unit are configured in a handheld electronic device while enabling the signal transceiver to transmit/receive the coordinate and the bio index through the communication network system and thus to display the same on the display unit.

8. The system of claim 6, wherein the handheld electronic device is designed to form a closed circuit with other handheld electronic devices in a manner that they are enabled to receive the coordinates and the bio indexes of different living entities from one another.

9. The system of claim 7, wherein the handheld electronic device is designed to form a closed circuit with other handheld electronic devices in a manner that they are enabled to receive the coordinates and the bio indexes of different living entities from one another.

10. The system of claim 4, wherein the calculation device is configured in a center operation system while enabling the same to receive the physiological attribute and then converting the received physiological attribute in to the bio index.

11. The system of claim 5, wherein the calculation device is configured in a center operation system while enabling the same to receive the physiological attribute and then converting the received physiological attribute in to the bio index.

12. The system of claim 6, wherein the handheld electronic device is a cellular phone.

13. The system of claim 7, wherein the handheld electronic device is a cellular phone.

14. The system of claim 4, wherein the calculation device and the display unit are configured in an electric calculation apparatus while enabling the calculation device to receive the coordinate and the physiological attribute from the communication network system and covert the received physiological attribute into the bio index and thus to display the bio index along with the coordinate on the display unit.

15. The system of claim 5, wherein the calculation device and the display unit are configured in an electric calculation apparatus while enabling the calculation device to receive the coordinate and the physiological attribute from the communication network system and covert the received physiological attribute into the bio index and thus to display the bio index along with the coordinate on the display unit.

16. The system of claim 14, wherein the electric calculation apparatus is designed to form a closed circuit with other electric calculation apparatuses in a manner that they are enabled to receive the coordinates and the bio indexes of different living entities from one another.

17. The system of claim 15, wherein the electric calculation apparatus is designed to form a closed circuit with other electric calculation apparatuses in a manner that they are enabled to receive the coordinates and the bio indexes of different living entities from one another.

18. The system of claim 14, wherein the electric calculation apparatus is a device selected from the group consisting of: a desktop computer and a notebook computer.

19. The system of claim 15, wherein the electric calculation apparatus is a device selected from the group consisting of: a desktop computer and a notebook computer.

20. The system of claim 1, wherein the physiological attribute is a value of physiological voltage.

21. The system of claim 1, wherein the bio index is an index adapted to be displayed as a chart selected from the group consisting of: an electroencephalogram (EEG), an electrocardiogram (ECG), an electrogastrogram (EGG) and an electromyogram (EMG).

22. The system of claim 1, wherein the living entity is a living thing selected from the group consisting of: a human being and an animal.

23. The system of claim 4, wherein the communication network system is a system selected from the group consisting of: a wireless communication system and a wireless network system.

24. The system of claim 5, wherein the communication network system is a system selected from the group consisting of: a wireless communication system and a wireless network system.

25. The system of claim 1, wherein the physiological status sensing device is designed to contact with the living entity for enabling the same to detect the physiological attribute of the living entity.

26. The system of claim 1, wherein the physiological status sensing device is designed to detect the physiological attribute of the living entity in a wireless sensing manner.

27. The system of claim 1, wherein the physiological status sensing device and the positioning device are configured in a physiological status monitoring/positioning apparatus to be used for detecting the physiological attribute of the living entity and the same time identifying the location of the living entity.

28. The system of claim 4, wherein the signals relating to the coordinate and the bio index are transmitted in an alternative manner representing the alternative relationship between the coordinate and the bio index.

29. The system of claim 5, wherein the signals relating to the coordinate and the bio index are transmitted in an alternative manner representing the alternative relationship between the coordinate and the bio index.

30. The system of claim 12, wherein the cellular phone further comprises an alert unit to be used for executing an alert operation as soon as the bio index exceeds a specific threshold value.

31. The system of claim 13, wherein the cellular phone further comprises an alert unit to be used for executing an alert operation as soon as the bio index exceeds a specific threshold value.

32. The system of claim 30, wherein the alert operation comprises the step of:
enabling the cellular phone to alerting those other cellular phones in the communication network system in a manner such as marking the cellular phone performing the alert operation as an eye-catching symbol on the display devices of those other cellular phones.

33. The system of claim 31, wherein the alert operation comprises the step of:
enabling the cellular phone to alerting those other cellular phones in the communication network system in a manner such as marking the cellular phone performing the alert operation as an eye-catching symbol on the display devices of those other cellular phones.

34. The system of claim 18, wherein there is an alert unit being configured in a device selected from the group consisting of: a desktop computer and a notebook computer, and to be used for executing an alert operation as soon as the bio index exceeds a specific threshold value.

35. The system of claim 19, wherein there is an alert unit being configured in a device selected from the group consisting of: a desktop computer and a notebook computer, and to be used for executing an alert operation as soon as the bio index exceeds a specific threshold value.

36. The system of claim 34, wherein the alert operation comprises the step of:
enabling the electric calculation apparatus to alerting those other the electric calculation apparatuses in the communication network system in a manner such as marking the electric calculation apparatus performing the alert operation as an eye-catching symbol on the display devices of those other the electric calculation apparatuses.

37. The system of claim 35, wherein the alert operation comprises the step of:
enabling the electric calculation apparatus to alerting those other the electric calculation apparatuses in the communication network system in a manner such as marking the electric calculation apparatus performing the alert operation as an eye-catching symbol on the display devices of those other the electric calculation apparatuses.

38. The system of claim 1, wherein the signal transceiver and the positioning device are integrated on a chip.

39. The system of claim 1, wherein the calculation device and the positioning device are integrated on a chip.
